(19) United States
(12) Patent Application Publication
(10) Pub. No.: US 2007/0212343 A1
(43) Pub. Date: Sep. 13, 2007

(54) COMPOSITIONS FOR TREATMENT OF EAR INFECTIONS

(76) Inventor: Geoffrey R. Owen, Southlake, TX (US)

Correspondence Address: ALCON, IP LEGAL, TB4-8, 6201 SOUTH FREEWAY, FORT WORTH, TX, 76134, US

(21) Appl. No.: 11/737,835

(22) Filed: Apr. 20, 2007

Related U.S. Application Data
(63) Continuation of application No. PCT/US05/33094, filed on Sep. 14, 2005.
(60) Provisional application No. 60/635,218, filed on Dec. 10, 2004.

(51) Int. Cl. A61K 38/48; A61K 31/4745

(52) U.S. Cl. 424/94.63; 424/94.64; 514/300

(57) ABSTRACT

Topical otic pharmaceutical compositions comprising moxifloxacin or a pharmaceutically useful hydrate or salt thereof and a proteolytic enzyme. The compositions facilitate trans-tympanic delivery of a therapeutic level of the moxifloxacin.

This application is a continuation of PCT/US2005/033094 filed Sep. 14, 2005 entitled “Compositions for Treatment of Ear Infections,” which claims priority from U.S. Provisional Application No. 60/635,218 filed Dec. 10, 2004.

### FIELD OF THE INVENTION

The present invention generally pertains to topical antibiotic pharmaceutical compositions for the treatment or prevention of otic infections and more particularly to moxifloxacin compositions for the treatment or prevention of middle or inner ear infections.

### DESCRIPTION OF THE RELATED ART

Nearly seventy percent of U.S. children develop otitis media (middle ear infection) by the age of two years. One half of such children have repeated episodes. Otitis media is the most common cause of hearing loss, and it often interferes with the childhood development and learning processes.

Systemic antibiotics are often prescribed for otitis media. Typical treatment regimens utilize systemic antibiotics for up to fourteen days, and side effects are quite common during this course of treatment.

Topical antibiotics are also available for treating otitis externa (external ear infection) and otitis media. Exemplary products include CIPRO® HC (ciprofloxacin hydrochloride equivalent to 0.2% ciprofloxacin; 1% hydrocortisone) otic suspension available from Alcon Laboratories, Inc. of Fort Worth, Tex.; CIPRODEX® (0.3% ciprofloxacin; 0.1% dexamethasone) otic suspension available from Alcon Laboratories, Inc.; and FLOXIN® (0.3% ofloxacin) otic solution available from Daiichi Pharmaceutical Corporation of Japan. CIPRO® HC otic suspension is indicated for treatment of acute otitis externa. CIPRODEX® otic suspension is indicated for the treatment of acute otitis externa and acute otitis media with tympanostomy tubes. FLOXIN® otic solution is indicated for the treatment of acute otitis externa, acute otitis media with tympanostomy tubes, and chronic suppurative otitis media with perforated tympanic membranes. However, it is generally accepted that conventional therapeutic agents do not cross the tympanic membrane. Therefore, existing products indicated for the middle ear all require the pre-placement of a tympanostomy tube via an outpatient surgical procedure or a pre-existing perforation of the tympanic membrane in order to deliver drug to the middle or inner ear. Accurate placement of drops through tympanostomy tubes can prove quite difficult in infants or small children who may be frightened of the ear drops and who are already agitated due to the pain of the infection.

U.S. Pat. No. 5,954,682 to Petrus discloses a therapeutic applicator 2 having a porous media 4 that may be soaked with one or more therapeutic agents. See '682 patent, FIG. 1; column 3, lines 59-61; column 6, lines 24-57. The '682 patent also discloses enzymatic and non-enzymatic penetration enhancers for allowing the infiltration of medications and chemical agents through the membranes and lining of the ear canal. See, e.g., '682 patent, column 6, line 58 through column 7, line 35. The '682 patent particularly discloses that “non-enzymatic penetration enhancers facilitate infiltration of biologically active agents, such as medications and chemical substances, through the membranes and lining of the ear canal 10”. '682 patent, column 7, lines 19-22.

The literature also reports that trypsin, a proteolytic enzyme, has been used in an ear drops solution containing tetracycline hydrochloride/polymixin B/betamethasone sodium phosphate and tetracaine. See “Otocusi Enzimatico” Brochure from Laboratories Cusi S-A, November 1992. The solution is used for treatment of purulent or nonpurulent painful inflammatory conditions of the external ear and middle ear. The brochure states that “trypsin is a proteolytic enzmyme that contributes to the destruction/elimination of necrotic tissue, pyogenic membranes, and incrustations”.

International Publication No. WO 03/003976, which is incorporated herein by reference, discloses a composition for assisting in the removal of human cerumen that includes a cerumenolytically acceptable enzyme that is useful in softening, dislodging, breaking-up, and/or digesting human cerumen in the external ear canal. Preferred cerumenolytically acceptable enzymes include include lipases, proteases, and amylases. Preferred proteases or proteolytic enzymes include pancreatin, trypsin, subtilisin, collagenase, keratinase, carboxypeptidase, papain, bromelain, aminopeptidase, elastase, Aspergillo peptidase, pronase E (from S. griseus), dispase (from Bacillus polymyxa) and mixtures thereof. See WO 03/003976, page 11, lines 1-19. The most preferred proteolytic enzyme is methyl trypsin. WO 03/003976, page 13, line 9.

U.S. Pat. No. 6,716,830, which is incorporated herein by reference, discloses the use of a potent new class of antibiotics to treat ophthalmic, otic and nasal infections, as well as the prophylactic use of these antibiotics following surgery or other trauma to ophthalmic, otic or nasal tissues. The disclosed compositions may also be administered to the affected tissues during ophthalmic, otic or nasal surgical procedures to prevent or alleviate post-surgical infection. The preferred antibiotic disclosed is moxifloxacin.

Improved moxifloxacin compositions that are effective for preventing or treating middle or inner ear infections, as well as methods of delivering such compositions, remain desirable.

### SUMMARY OF THE INVENTION

The present invention comprises topical otic pharmaceutical compositions comprising moxifloxacin or a pharmaceutically useful hydrate or salt thereof and a proteolytic enzyme. The compositions facilitate trans-tympanic delivery of a therapeutic level of the moxifloxacin.

### DETAILED DESCRIPTION OF THE PREFERRED EMBODIMENTS

Unless otherwise indicated, all ingredient concentrations listed as a percentage are in units of weight/volume percent.

The preferred composition of the present invention includes moxifloxacin or its pharmaceutically useful hydrates and salts and a penetration enhancer to facilitate the delivery of the moxifloxacin across an intact tympanic membrane. Further details regarding the structure, preparation, and physical properties of moxifloxacin are provided in U.S. Pat. No. 5,607,942, which is incorporated herein by reference. Moxifloxacin is preferably present in the amount of 0.1-1%, and most preferably 0.5%. Preferred penetration enhancers include proteolytic enzymes such as trypsin, collegenase, and pepsin. Trypsin is preferably present in the amount of 0.1-10 mg/ml, and most preferably 5 mg/ml. Collagenase is preferably present in the amount of 0.1-10 mg/ml, and most preferably 5 mg/ml. Pepsin is preferably present in the amount of 0.1-10 mg/ml, and most preferably 5 mg/ml. A preferred buffer solution comprises sodium acetate.3H₂O, sodium chloride, calcium chloride.2H₂O, water, and a pH adjuster. The preferred pH adjusters are sodium hydroxide or hydrochloric acid. Sodium acetate.3H₂O is preferably present in the amount of 0.1-1%, and most preferably 0.68%. Sodium chloride is preferably present in the amount of 0.1-1%, and most preferably 0.60%. Calcium chloride.2H₂O is preferably present in the amount of 0.01-1%, and most preferably 0.05%. Water is added in a quantity sufficient to result in a desired volume. The pH adjuster is added in quantity sufficient to bring the pH of the composition to 6-8, and most preferably 7.5.

It has been unexpectedly discovered that in such a composition, proteolytic enzymes are superior penetration enhancers for trans-tympanic delivery of moxifloxacin than conventional skin penetration enhancers, such as dimethylsulfoxide (“DMSO”) and purified diethlene glycol monoethyl ether (Transcutol® P). In addition, it has been unexpectedly discovered that collegenase is a superior penetration enhancer for trans-tympanic delivery of moxifloxacin than trypsin or pepsin.

The compositions of the present invention are specially formulated for topical application to otic tissues. The compositions are preferably sterile and have physical properties that are specially suited for application to otic tissues, including tissues that have been compromised as the result of preexisting disease, trauma, surgery or other physical conditions.

The compositions of the present invention are preferably packaged in a bottle that may be squeezed to dispense a drop(s) of the composition within a user's ear via a nozzle, or a bottle having a pump that may be actuated to deliver a spray(s) of the composition within a user's ear. Certain preferred devices for dispensing the compositions of the present invention are described in U.S. Pat. Nos. 5,474,209 and 5,782,345, and in International Publication No. WO 03/003976, which are incorporated herein by reference.

Otic pharmaceutical products are typically packaged in multidose form. Preservatives may be required to prevent microbial contamination during use. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically such preservatives are employed at a level of from 0.001% to 1.0%.

The solubility of the components of the present compositions may be enhanced by a surfactant or other appropriate co-solvent in the composition. Such co-solvents include polysorbate 20, 60, and 80; polyoxyethylene/polyoxypropylene block copolymer surfactants (e.g., Pluronic® F-68 and Tetronic® 1304); cyclodextrin; or other agents known to those skilled in the art. Typically such co-solvents are employed at a level of from 0.01% to 2%.

The use of viscosity enhancing agents to provide the compositions of the invention with viscosities greater than the viscosity of simple aqueous solutions may be desirable to increase the retention time in the ear. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, or other agents know to those skilled in the art. Such agents are typically employed at a level of from 0.01% to 2%.

The following example is intended to illustrate, but in no way limit, the present invention.

### EXAMPLE 1

A laboratory model was developed using discs of New Zealand White rabbit ear harvested from close to the tympanic membrane. The discs are supported in a simple apparatus that mimics the anatomy of the ear. The inner skin side of the rabbit ear is the “donor side”, and the fur side of the rabbit ear, with the skin layer removed, is the “acceptor side”. 0.5% moxifloxacin was chosen as the therapeutic agent for the example along with various penetration enhancers. The moxifloxacin and one of the penetration enhancers were added to an acetate buffer solution having 0.68% sodium acetate.3H₂O, 0.60% sodium chloride, 0.05% calcium chloride.2H₂O, q.s. water, and q.s. pH adjuster to bring the pH of the composition to 7.5. The resulting compositions were applied to the donor side of the discs. Table 1 shows the level of moxifloxacin in the buffer on the acceptor side of the discs as measured via HPLC after 2 hours.

TABLE 1

| | Concentration of Moxifloxacin |
|---|---|
| 0.5% Moxifloxacin + | on the Acceptor Side of Rabbit Ear |
| Acetate Buffer Solution | 0.17 ppm |
| DMSO 10% + Acetate Buffer | 0.22 ppm |
| Solution | |
| Transcutol ® P 50% + Acetate | 0.49 ppm |
| Buffer Solution | |
| Trypsin 5 mg/ml + Acetate | 0.93 ppm |
| Buffer Solution | |
| Collagenase 5 mg/ml + Acetate | 1.81 ppm |
| Buffer Solution | |
| Pepsin 5 mg/ml + Acetate | 1.51 ppm |
| Buffer Solution | |

Middle ear fluid concentration of antibiotics during systemic dosing (e.g. oral dosing @ 500 mg/day) are typically in the range of 1 to 10 ppm, depending on the particular antibiotic. Typically, the MIC levels for moxifloxacin are 1 to 2 ppm for many of the bacteria commonly associated with otic infections. DMSO and Transcutol® P were chosen as typical conventional skin penetration enhancers. Both DMSO and Transcutol® P are non-enzymatic penetration enhancers. As shown by Table 1, compositions of moxifloxacin, a non-enzymatic penetration enhancer (e.g. DMSO or Transcutol® P), and buffer do result in higher concentrations of moxifloxacin on the acceptor side of the rabbit ear than compositions of moxifloxacin and buffer alone. However, compositions of moxifloxacin, an enzymatic penetration enhancer (e.g. trypsin, collagenase, or pepsin) unexpectedly result in significantly higher concentrations of moxifloxacin at or above the MIC level for moxifloxacin for many bacteria commonly associated with otic infections. Such concentrations of moxifloxacin represent therapeutic levels.

From the above, it may be appreciated that the present invention provides improved moxifloxacin compositions that are effective for preventing or treating middle or inner ear infections, as well as methods of delivering such compositions. It is believed that the operation and construction of the present invention will be apparent from the foregoing description. While the compositions and methods shown or described above have been characterized as being preferred, various changes and modifications may be made therein without departing from the spirit and scope of the invention as defined in the following claims.

What is claimed is:

1. A topical otic pharmaceutical composition comprising moxifloxacin or a pharmaceutically useful hydrate or salt thereof and a proteolytic enzyme.

2. The composition of claim 1 wherein said proteolytic enzyme is trypsin.

3. The composition of claim 1 wherein said proteolytic enzyme is collagenase.

4. The composition of claim 1 wherein said proteolytic enzyme is pepsin.

5. The composition of claim 1 wherein said composition facilitates trans-tympanic delivery of a therapeutic level of said moxifloxacin.

6. The composition of claim 1 further comprising an acetate buffer.

7. A topical otic pharmaceutical composition comprising:
moxifloxacin or a pharmaceutically useful hydrate or salt thereof having a concentration of moxifloxacin of 0.1-1%; and
trypsin in a concentration of 0.1-10 mg/ml.

8. The composition of claim 7 wherein said composition facilitates trans-tympanic delivery of a therapeutic level of said moxifloxacin.

9. The composition of claim 7 further comprising an acetate buffer.

10. A topical otic pharmaceutical composition comprising:
moxifloxacin or a pharmaceutically useful hydrate or salt thereof having a concentration of moxifloxacin of 0.1-1%; and
collagenase in a concentration of 0.1-10 mg/ml.

11. The composition of claim 10 wherein said composition facilitates trans-tympanic delivery of a therapeutic level of said moxifloxacin.

12. The composition of claim 10 further comprising an acetate buffer.

13. A topical otic pharmaceutical composition comprising:
moxifloxacin or a pharmaceutically useful hydrate or salt thereof having a concentration of moxifloxacin of 0.1-1%; and
pepsin in a concentration of 0.1-10 mg/ml.

14. The composition of claim 13 wherein said composition facilitates trans-tympanic delivery of a therapeutic level of said moxifloxacin.

15. The composition of claim 13 further comprising an acetate buffer.
